(19) United States
(12) Patent Application Publication
(10) Pub. No.: US 2017/0056879 A1
(43) Pub. Date: Mar. 2, 2017

(54) FIELD PATHOGEN IDENTIFICATION

(71) Applicant: BG RESEARCH LTD, Cambridgeshire (GB)

(72) Inventors: Nelson Nazareth, Upper Dean (GB); David Edge, Warlingham (GB); Adam Tyler, Burton Latimer (GB)

(21) Appl. No.: 15/330,058

(22) PCT Filed: Jan. 28, 2015
(86) PCT No.: PCT/GB2015/000027

§ 371 Date: Jul. 29, 2016

(30) Foreign Application Priority Data
Jan. 29, 2014 (GB) 1401584.6

(51) Int. Cl. B01L 3/00; B01L 9/00; B01L 7/00; G01N 21/64; C12Q 1/68

(52) U.S. Cl. CPC B01L 3/50851; G01N 21/6456; G01N 21/6428; C12Q 1/686; B01L 7/52; B01L 9/523; G01N 2021/6484; B01L 2300/1822

(57) ABSTRACT

A process for the identification of genetic material in a biological liquid sample and including the steps of injecting into a reaction vessel containing freeze dried PCR reagents and labelled primers sufficient pure water to liquify the reagents and primers; injecting the biological liquid sample into the reaction vessel; subjecting the reaction vessel contents to a cell disruption process, in the said reaction vessel; conducting pathogen specific polymerase chain reaction (PCR) on the contents of the reaction vessel; and monitoring the PCR and determining therefrom the presence of a specific genetic material. A device for performing the process comprises a heat reduction module (HRM); a peltier cell (TEC) the base plate whereof is contiguous with the HRM; a reaction vessel receiving heat transfer sleeve contiguous with a working face of the TEC; retention means for holding a reaction vessel in the sleeve; and means for driving the TEC.

### FIELD OF THE INVENTION

The present invention relates to the identification of pathogens which may be present in human or animal blood. It is particularly concerned with the rapid field identification of dangerous pathogens if found in a blood sample.

### SUMMARY OF THE INVENTION

According to a first aspect of the invention there is provided a device for carrying out a rapid field identification of a pathogen, the device comprising:
a heat reduction module (HRM);
a pettier cell (thermos-electric cell) (TEC) the base plate whereof is contiguous with, and preferably attached to, the HRM;
a reaction vessel receiving heat transfer sleeve contiguous with, and preferably attached to, a working face of the TEC;
retention means arranged for holding a reaction vessel in the sleeve;
means for driving the TEC; and
optical means arranged For monitoring a reaction in the reaction vessel
and the device arranged for operation with a microtitre reaction vessel and a holder therefor, the holder being formed to retain the reaction vessel and to receive the sleeve and fit retentively to the retention means.

The preferred microtitre reaction vessel is one which is formed of a carbon loaded plastics material. With such a reaction vessel heat can rapidly be transferred into and out of the reaction chamber. Such a microtitre vessel may be of the order of 2cm overall length and comprise, in descending order, a cap receiving rim, a filler portion, a reaction chamber with a base thereto. The filler portion may have a maximum outer diameter of 7-8 mm and a depth of about 4-5 mm and the reaction chamber tapering down from 3 mm to 2.5 mm diameter, the whole having a wall thickness of the order of 0.8 mm. Accordingly the reaction vessel may be of substantially capillary dimensions in order to maximize the rates of heat transfer. The reaction vessel will normally have a transparent lid sealable thereto.

Because manipulation of a microtitre vessel will be fiddly for a gloved manual worker it is preferably supplied fitted to an individual holder somewhat in the form of a top hat and arranged to be placeable on a flat surface separate from the heater part of the device for charging and brought thereto for processing. This individual holder may have a tag formed thereon carrying the removable vessel lid. Thus the retention and fitting of the lid is facilitated. Formed of a plastics material the well, and the reaction vessel still held therein can be readily disposable upon completion of the identification process.

A preferred thermocycler device comprises a metal sleeve adapted snugly to surround the reaction vessel reaction chamber in such a manner as to be contiguous therewith throughout the length thereof and, integral with the sleeve at the base thereof, a heat transfer foot. The heat transfer foot is advantageously attached to the working face of a Peltier cell the base face whereof is attached to a heat reduction module (HRM) arranged for operation around a median temperature, that temperature being typically around the annealing temperature of an average DNA. The top hat advantageously receives the sleeve. In the base of the sleeve, adjacent the TEC, may be a thermistor enabling the temperature of the process to be continually monitored and controlled. The sleeve may be formed of a metal which is both a good heat conductor and relatively resistant to corrosion, such as brass. To minimise the possibility of delamination of the Peltier cell itself or its detachment from either the HRM or the heat transfer foot the attachment may be effected with a flexible solder such as lead tin solder 183.

The heat reduction module (HRM) may be a device arranged for the flow therethrough of a heat transfer liquid supplied thereto at constant temperature. For simplicity in the field context the liquid may be water. There may be a reservoir containing a heating element and/or fan arranged to keep the coolant at a constant temperature, and an associated pump.

There may be a fixing block arranged to surround and protect the TEC and the sleeve and also to act as a mount for the reaction vessel holder. The arrangement can be accordingly such that “putting on the top hat” fits the holder retentively to the fixing block and brings the reaction vessel into a snug fit in the sleeve.

The device may further comprise a pure water injection station arranged for dispensing a metered quantity of pure water into the reaction vessel when the latter contains freeze dried materials. Likewise or alternatively there may be a reagent dispenser arranged for dispensing such reagents as are not freeze dried.

According to a further aspect of the invention there is provided a microtitre reaction vessel and a holder therefor, the holder being formed to retain the reaction vessel and to receive the sleeve and fit retentively to the fixing block.

For the microtitre context in particular the optical arrangement is also as miniaturised as possible. The optical arrangement normally comprises an excitation light source arranged for exciting fluorescence in the reaction chamber and a collector of emitted fluorescence and conveyance thereof to a spectrophotometer. The excitation source and perhaps the excited light collector may be adjacent or close to the reaction vessel lid. The collector is preferably an optic fibre. In an alternative embodiment the optic fibre may be bifurcated or have two or more cores, with one core arranged for excitation and another for collection.

The light source may comprise a laser diode or light emitting diode (LED) or a broad spectrum source halogen lamp filtered to the required emission wavelength. The preferred optical reader is a spectrophotometer but a photodiode may be used.

Typically a station comprising the HRM, the TEC, the sleeve and the fixing block may be one station in an array. For rapid field operation this station advantageously also has an optical unit comprising a source and collector. This optical unit is very preferably arranged to be movable between operation and access positions. Hinge means may be provided for this, advantageously organized to minimise interruption of the optical paths, including bending of the optic fibre(s). The optical unit may further have retention means, such as magnets, to ensure a degree of locking in the operational and access positions. In the operational position the retention means preferably acts to retain the unit against the reaction vessel lid, and may include a lid heater.

For field operation the array may comprise eight stations and accordingly be attached to a field operation board on a pathogen detection unit which can also comprise power supplies and controls and the optical system. The array is preferably organised for individual control of each station, while the HRMs are in communication one with another via a common heater and pump. Also the unit is preferably constructed so that expensive electronic and optical equipment are sealed in a container the exterior of which can be repeatedly, thoroughly and safely sterilised without affecting deleteriously the content thereof.

An advantage of such an arrangement is that it can readily be portable and operated from the auxiliary 12 volt battery of a four wheel drive vehicle such as a Landrover.

According to a second aspect of the present invention a process for the identification of genetic material in a biological liquid sample comprises the steps of:
injecting into a reaction vessel containing PCR reagents and labelled primers a biological liquid sample into the reaction vessel;
if necessary subjecting the reaction vessel contents to a cell disruption process, in the said reaction vessel;
conducting pathogen specific polymerase chain reaction (PCR) on the contents of the reaction vessel;
monitoring the PCR and determining therefrom the presence of a specific genetic material.

Preferably the reaction vessel contains the PCR reagents and labelled primers in freeze dried form and a first step of the process comprises placing in the vessel sufficient pure water to liquefy the reagents and primers. Preferably also the monitoring is performed in real time, employing fluorescence.

Ideally the reaction vessel is of microtitre proportions. This has the particular advantage that when for example a sample of blood is collected using a fixed volume capillary aspirant/dispenser such as a “MICROSAFE”, exactly the required quantity of the blood can be delivered to the reaction vessel. Not only that but then the collection and delivery can readily be performed by a health worker wearing protective clothing.

It will accordingly be appreciated that the invention is particularly intended to assist in the rapid identification of extremely dangerous pathogens, such as the ebola virus.

The cell disruption process may comprise one or both of freezing then thawing and boiling then cooling the sample so the genetic material cells therein are broken open. This cyclic operation may be performed a few times, for example up to five cycles.

According to a feature of the invention the cell disruption and the PCR may be carried out employing the same reaction vessel and heating/cooling means. A suitable heating/cooling means comprises a Peltier cell, hereinafter called a TEC (thermoelectric cell) arranged to operate against a constant temperature, advantageously one intermediate the freezing and boiling temperatures of the sample. Typically the said intermediate temperature is around the annealing temperature of an average DNA. This intermediate, constant temperature may be supplied by a Heat Reduction Module (HRM) as described above.

Contiguous with a working face of the TEC may be a heat transfer sleeve arranged to receive snugly a reaction vessel.

Monitoring the PCR process is preferably performed by reader means comprising an optical arrangement, typically incorporating a spectrophotometer relying on LED excitation and CCD detection. The process can then include comparison of the resultant spectrum with that of a target DNA.

Typically the dimensions of the device are a length of the order of 50 mm and a diameter of 55 mm. This is consistent with ready manual handling by a protective suited operative on the one hand and cheap manufacture for disposability on the other. A suitable Peltier cell for such a device measures 9 mm square.

It is a particularly important feature of devices and processes in accordance with the present invention that the process can be completed within ten minutes, in other words whilst a patient is waiting.

### BRIEF DESCRIPTION OF THE DRAWINGS

A process and device embodiment will now be described by way of example with reference to the accompanying drawings, of which:

FIG. 1 is an exploded view of the device;

FIG. 2 is a section of a microtitre reaction vessel and holder therefor;

FIG. 3 is a section of an assembled device;

FIG. 4 is an isometric view of an eight station array unit.

FIGS. 5 and 6 are exploded views of the eight station array unit;

FIGS. 7, 8 and 9 illustrate an optical unit; and

FIG. 10 illustrates the array unit control panel and screen.

### SPECIFIC DESCRIPTION

As shown in the figures a device for the identification of a pathogen comprises a heat reduction module HRM, a peltier cell TEC, a heat transfer sleeve 30, retention means 40, a microtitre reaction vessel 50 and a vessel holder 60.

The heat reduction module HRM comprises a vessel 10 with a lid 11 sealable thereto. The vessel 10 has liquid entry and exit ports 12 and 13 and the lid has a nest 14 for the accurate and economic reception of the TEC.

The TEC has a base face 20 attached to the lid 11 within the nest 14, and a working face 21.

The sleeve 30 is formed of brass and comprises a reception portion 31 and a heat transfer foot 32. The reception portion 31 is adapted to receive snugly the reaction chamber portion 51 of a reaction vessel 50. The foot 32 is formed with the same sole diameter as the TEC working face 21 breadth to which it is attached. Attached to the sleeve 30 is a thermistor 33.

A PCB 15 mounted to the lid 11 connects the TEC and the thermistor 33 to supply and control circuits.

The retention means 40 comprises a base plate 41 and a cover 42 attached to the base plate 41. The base plate 41 is attached to the HRM lid 11. The function of the retention means 40 is to house and protect the sleeve 30 and to receive the reaction vessel 50 and its holder 60.

The reaction vessel 50 is a microtitre vessel formed of a carbon loaded plastics material and is 2 cm overall length. It comprises, in descending order, a cap receiving rim 51, a filler portion 52 and a reaction chamber 53 with a base 54 thereto. The filler portion 52 has a maximum outer diameter of 7 mm and a depth of 5 mm. The reaction chamber 53 tapers down from 3 mm to 2.5 mm, the whole having a wall thickness of 0.8 mm. Accordingly the reaction vessel 50 is of substantially capillary dimensions.

The holder 60 receives and retains a reaction vessel 50. It is shaped as a top hat and thus receives the sleeve 30. Formed on the holder 60 is a flexible tag 61 carrying, at a distal end thereof, a transparent lid 62 sealable to the rim 51 of the reaction vessel 50.

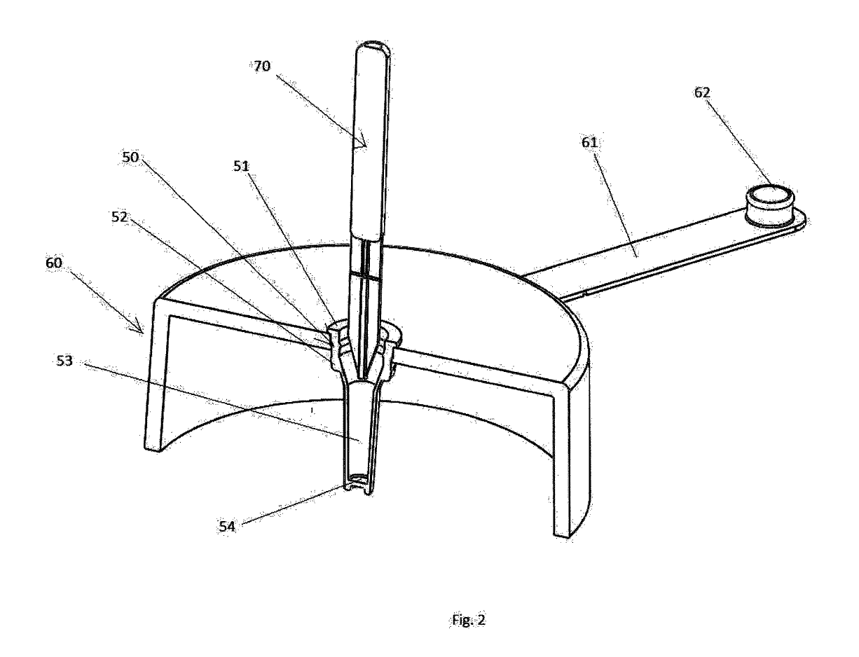

The vessel 50 is, as shown in FIG. 2, adapted for the reception of a sample from a fixed volume capillary aspirator/dispenser (MICROSAFE) 70. This in turn can receive its sample from a standard blood extraction device.

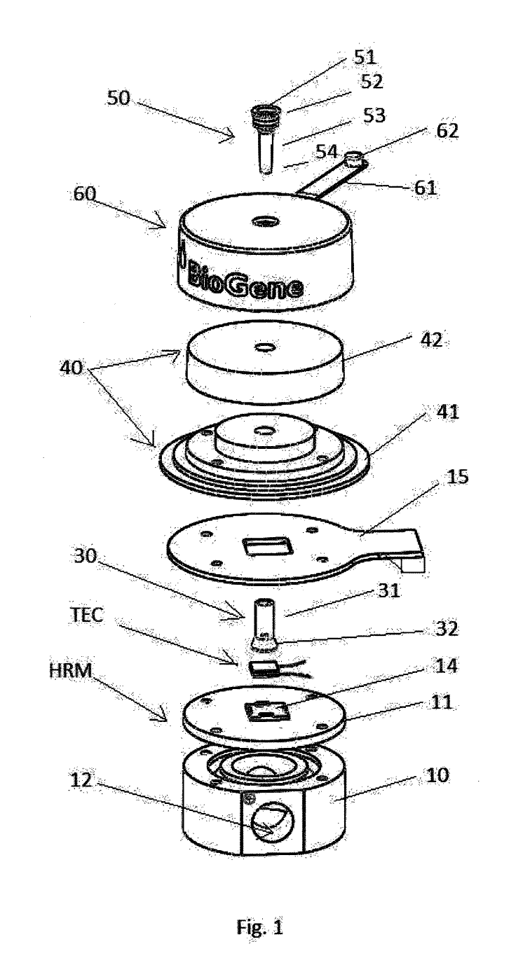

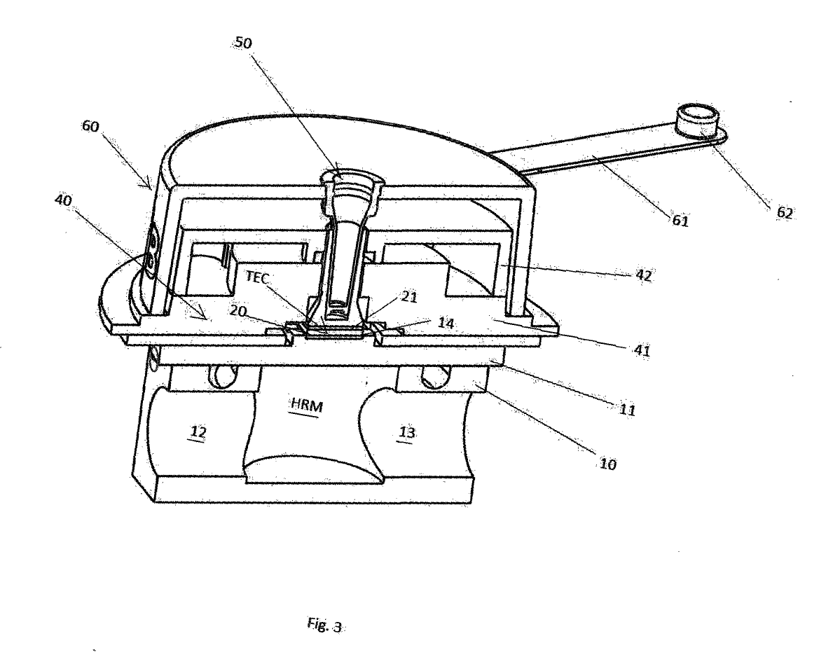

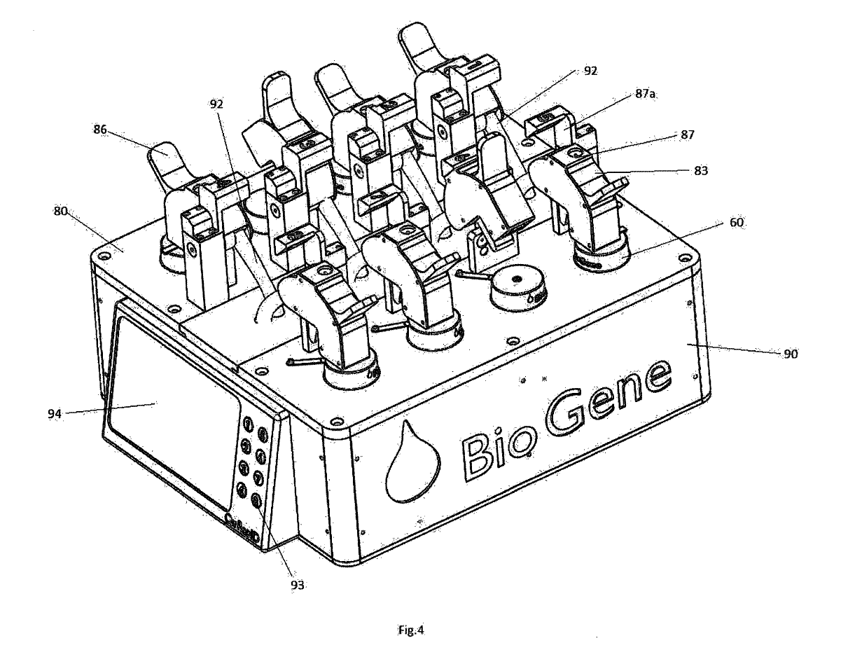

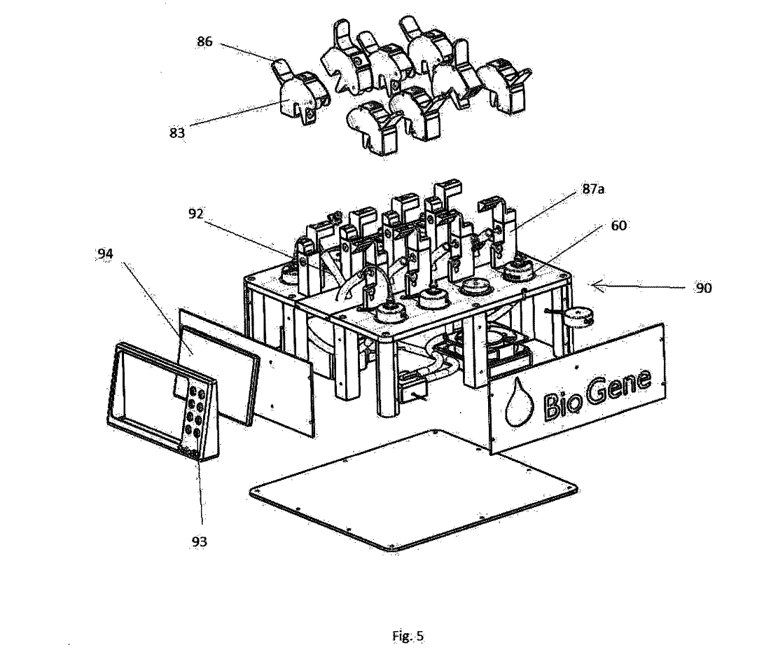

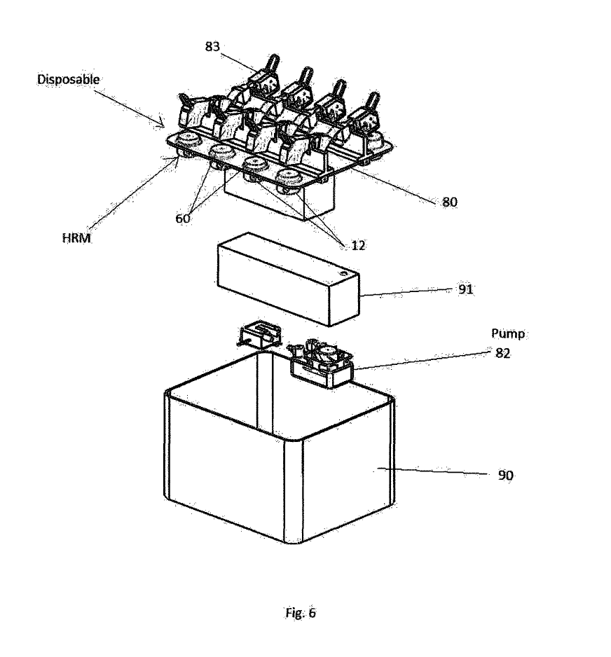

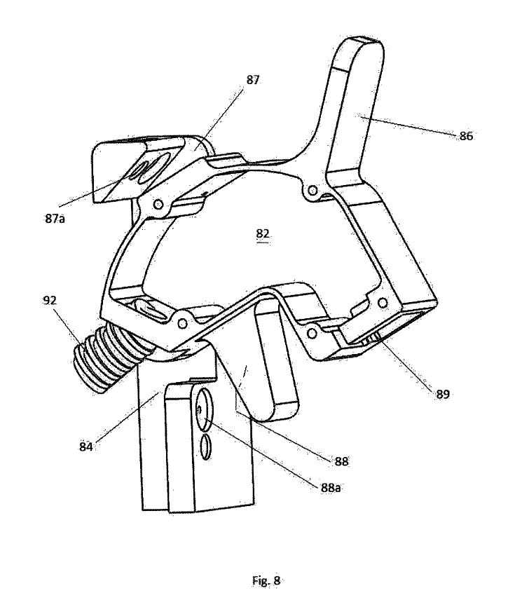

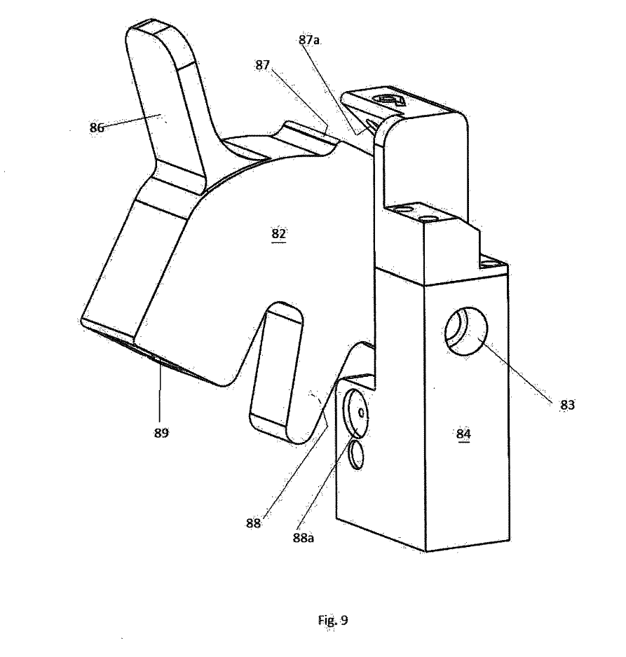

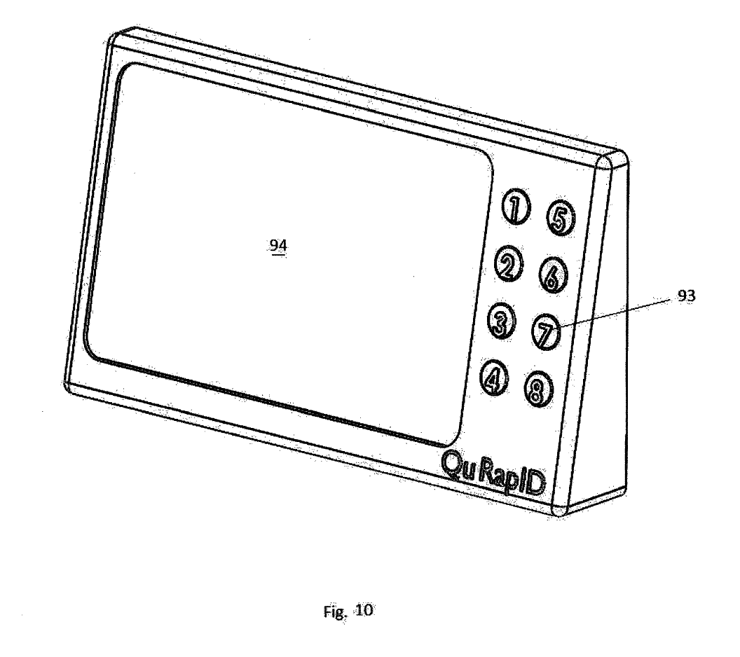

Shown schematically in FIGS. 4 to 10 is one embodiment of an array of the identification devices depicted in FIGS. 1 to 3. Fitted to an array board 80 are eight such devices. The cover 42 of the retention means or, when fitted, the holders 60 can be seen above the board 80. Below the board 80 is the HRM. The inlet 12 and outlet 13 of the HRM communicate with those of the other devices, also with a heater and pump unit 81, whereby liquid can be circulated through the HRMs of the devices continuously, at a constant temperature. The board 80 is mounted to a case 90.

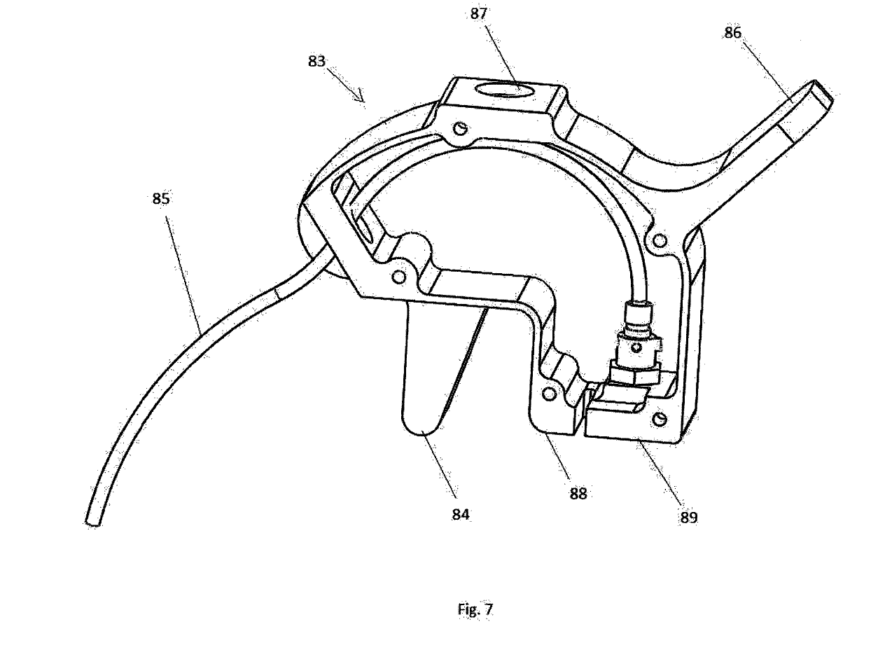

The optical arrangement comprises an optical unit 82 hinged at position 83 to a pillar 84 mounted on the array board 80 and a spectrophotometer mounted in the case 90. The optical unit 82 Is particularly illustrated in FIGS. 7 to 9 and contains a bifurcated optic fibre cable 85 in which one core is the exciting fibre and the other the collector. The position of the hinge 83 is such as to minimise disturbance of the cable 85.

The optical unit 82 is movable between operation and access positions by a toggle 86. The optical unit 82 has retention magnets 87 and 88 arranged hold the unit 82 in the access and operational positions respectively. Corresponding magnets 87a and 88a on the pillars 84 serve to detain the magnets 87 and 88. The unit 82 also incorporates a lid heater 89 and associated sensor so that In the operational position the retention means acts to retain the lid heater 89 against the lid 62, to prevent misting thereof.

Within the case 90 is a sealed box 91 containing the device electronic equipment, the light source and the collected light reader. These are thus protected from access by spilled pathogens while the exterior of the box 91 can be sterilised. A flexible duct 92 communicates between the sealed box 91 and the optical unit 83, sealed to each and containing the electrical feed for the heater 89 and the optic cable 85.

In an alternative embodiment the light source and/or light sensor is mounted in the unit 82. In another alternative embodiment the device incorporates a station arranged for delivering a dose of pure water into a reaction vessel, thus to liquefy freeze dried reagents. In yet another embodiment the device incorporates a station arranged for delivery of reagents into a reaction vessel.

Upon the case 90 is mounted a display panel 93, with a touch screen 94. The touch screen 94 comprises the means by which an operative initiates and controls the process in any one device and observes the outcome of the process. The display panel comprises one LED unit for each device and provides a simple indication that a particular device is in use and the progress of the process therein.

The process for the identification of genetic material in a blood sample and employing the device comprises the steps of:
injecting into a microtitre reaction vessel as described above and containing freeze dried PCR reagents and labelled primers a sufficient quantity of pure water
injecting into the reaction vessel a biological liquid sample;
if necessary subjecting the reaction vessel contents to a cell disruption process, in the said reaction vessel;
conducting pathogen specific polymerase chain reaction (PCR) in the reaction vessel on the contents thereof the reaction vessel; and
monitoring the PCR and determining therefrom the presence of a specific genetic material.

The blood sample will have been collected using a fixed volume capillary aspirant/dispenser such as a “MICROSAFE”, whereby exactly the required quantity of the blood can be delivered to the reaction vessel. With a device as described herein this collection and delivery can readily be performed by a health worker wearing protective clothing.

For blood the cell disruption process comprises boiling then cooling the sample so that the genetic material cells therein are broken open. This cyclic operation may be performed a few, for example up to five times.

With the base face 20 of the TEC 14 held at a constant temperature slightly above the annealing temperature of the average DNA/RNA, a positive current to the working face 21 causes that face 21 to heat to a temperature above that of the base face 20. A negative current supplied to the working face 21 causes the temperature thereof to sink to below that of the base face 20.

The PCR process is monitored by reader means comprising an optical arrangement typically incorporating a spectrophotometer relying on LED excitation and CCD detection. The resultant spectrum is then compared with that of a target DNA/RNA and whether or not the sample contained the target DNA is thus determined.

Upon completion of the process in any particular device the holder 60, carrying the sealed reaction vessel 50 can be removed from the board 80/device and discarded, preferably incinerated.

Typical dimensions of the device are a length of 40 mm and a diameter of 55 mm. This is consistent with ready manual handling by a protective suited operative on the one hand and cheap manufacture for disposability on the other. It means that the holder 60 can be about 55 mm diameter and 2 cm in depth. A suitable Peltier cell for such a device measures 9 mm square. The overall dimensions of the case 90, including the optical units are 55 cm long×45 cm broad×35 cm deep.

Although the device and process have been described as suitable for detecting a target DNA/RNA in blood, they can readily be used to detect target DNA/RNA in urine or saliva.

1-53. (canceled)

54. A device for carrying out a process of rapid field identification of a pathogen, the device comprising:
a heat reduction Module (HRM);
a peltier cell having a base face and a working face, the base face being contiguous with the heat reference module,
a reaction-vessel-receiving heat transfer sleeve contiguous with the peltier cell working face;
retention means for holding a reaction vessel in the sleeve;
means for driving the peltier cell, said means including an electrical supply and a temperature sensor;
means for monitoring the process and indicating the result thereof; and
and the device being arranged for operation with a microtitre reaction vessel and a holder therefor, the holder being formed to retain the reaction vessel and to receive the sleeve and fit retentively to the retention means.

55. A device as claimed in claim 54 and wherein the means for monitoring the process and indicating the result thereof comprises an optical unit.

56. A device as claimed in claim 55 and wherein the optical unit comprises a bifurcated optic fibre cable, an LED excitation unit and a spectrophotometer.

57. A device as claimed in claim 54 and wherein the peltier cell base face is attached to the HRM and the sleeve is attached to the peltier cell working face.

58. A device as claimed in claim 54 and wherein the heat transfer sleeve is adapted snugly to surround a reaction vessel reaction chamber.

59. A device as claimed in claim 54 and further comprising a reservoir containing a heating element arranged to keep heat transfer liquid at a constant temperature, and an associated pump.

60. A device as claimed in claim 54 and wherein the retention means comprises a fixing block arranged to surround and protect the peltier cell and the sleeve and also to act as a mount for the reaction vessel.

61. A device as claimed in claim 54 and arranged to be driven by a 12 volt supply.

62. A device as claimed in claim 54 and wherein the heat reference module, the peltier cell, the sleeve and the retention means form a station in a multiple array thereof.

63. A device as claimed in claim 62 and arranged for individual operation and control of the individual stations independently one of another.

64. A device for carrying out a process of a rapid field identification of a pathogen, the device comprising an array of stations arranged for individual control, each incorporating:
a heat reference module, arranged for the flow therethrough of water supplied thereto at constant temperature and having an associated reservoir containing a heating element arranged to keep the water at a constant temperature, and a pump;
a peltier cell having a base face and a working face, the base face being contiguous with the heat reference module;
a reaction vessel receiving heat transfer sleeve contiguous with the peltier cell working face, the sleeve arranged to receive snugly and surround a microtitre reaction vessel reaction chamber.
retention means for holding a reaction vessel in the sleeve and comprising a fixing block arranged to surround and protect the peltier cell and the sleeve and also to act as a mount for the reaction vessel;
electrical means arranged for driving the peltier cell;
a thermistor arranged to enable the temperature of the process to be continually monitored and controlled; and
an optical unit having a bifurcated optic fibre cable, an LED excitation unit and a spectrophotometer.

65. A device as claimed in claim 64 and arranged for operation with a reaction vessel of the order of 2 cm overall length and comprising, in descending order, a cap receiving rim, a filler portion, a reaction chamber with a base thereto, the filler portion having a maximum outer diameter of 7-8 mm and a depth of about 4-5 mm and the reaction chamber tapering down from 3 mm to 2.5 mm, the whole having a wall thickness of the order of 0.8 mm.

66. A process for the identification of genetic material in a biological liquid sample and comprising the steps of:
injecting the biological liquid sample into a microtitre reaction vessel containing PCR reagents and labelled primers;
placing the reaction vessel in a device as claimed in claim 1;
subjecting the reaction vessel contents to a cell disruption process, in the said reaction vessel;
conducting pathogen specific polymerase chain reaction (PCR) on the contents of the reaction vessel; and
monitoring the PCR and determining therefrom the presence of a specific genetic material.

67. A process as claimed in claim 66 and wherein PCR reagents and labelled primers are freeze dried and a first step comprises placing in the vessel sufficient pure water to liquefy the reagents and primers.

68. A process as claimed in claim 66 and wherein the cell disruption process comprises freezing then thawing and boiling then cooling the sample so the genetic material cells therein are broken open.

69. A process as claimed in claim 68 and wherein the PCR is then carried out employing the same reaction vessel in the same device.

70. A process as claimed in claim 64 and wherein the said constant temperature is intermediate the freezing and boiling temperatures of the sample.

71. A process as claimed in claim 70 and wherein the said intermediate temperature is that of the local environment.

72. A process as claimed in claim 64 and wherein monitoring the PCR process is performed by reader means comprising an optical arrangement.

73. A process as claimed in claim 72 and wherein the optical arrangement incorporates a spectrophotometer relying on LED excitation and CCD detection and the process further comprises comparison of the resultant spectrum with that of a target DNA.
